(19) United States
(12) Patent Application Publication
(10) Pub. No.: US 2006/0020259 A1
(43) Pub. Date: Jan. 26, 2006

(54) System for performing a corneal transplantation

(76) Inventors: Klaus Baumeister, Sinsheim-Adersbach (DE); Tobias Kuhn, Heidelberg (DE); Frieder Loesel, Mannheim (DE); Fritz Meisel, Kalchreuth (DE)

Correspondence Address: NEIL K. NYDEGGER;NYDEGGER & ASSOCIATES, 348 Olive Street, San Diego, CA, 92103, US

(21) Appl. No.: 10/895,276

(22) Filed: Jul. 20, 2004

(51) Int. Cl. A61F 9/009; A61B 18/20; A61F 9/01

(52) U.S. Cl. 606/5; 606/107

(57) ABSTRACT

A system for performing a corneal transplantation includes a laser source for generating a laser beam and a chair for positioning a patient relative to the laser source. A stabilizing element, engageable with the laser source, is fixated on the anterior surface of the patient's cornea to hold the cornea in alignment with the laser source. The laser source is then used to remove diseased tissue from the cornea of the patient, thereby creating a corneal cavity of known dimensions. In a subsequent step, a donor graft that was previously photoaltered to have substantially the same dimensions as the corneal cavity, is transplanted into the corneal cavity.

### FIELD OF THE INVENTION

The present invention pertains generally to systems and methods for performing ophthalmic laser surgery. More particularly, the present invention pertains to surgical procedures for performing a corneal transplantation wherein a donor graft and the cavity in the cornea of a patient for receiving the graft have the same dimensions. The present invention is particularly, but not exclusively, useful as a system for using a laser source to create a corneal cavity and a donor graft having a same geometry.

### BACKGROUND OF THE INVENTION

A corneal transplantation procedure (keratoplasty) involves replacing the diseased or damaged tissue of a patient's cornea with a graft of healthy tissue that is taken from a donor cornea. In such a procedure, it is obviously desirable that the donor graft be as near the same size and shape as the volume of tissue that is being replaced. It happens, however, that corneal transplantation procedures do not routinely achieve this objective.

Corneal transplantation procedures have been generally performed using either a knife or some form of laser procedure to prepare the patient's cornea and create a donor graft. Heretofore, regardless how the procedure has been performed, several factors have conspired to complicate matters. In particular, for procedures wherein a knife (e.g. a trephine) has been used to prepare a patient's cornea for a corneal transplantation, two issues commonly arise. First, the proper positioning and stabilization of the patient's eye during the procedure has always been difficult. Indeed, in such procedures it is typically necessary for the eye to be physically grasped (e.g. use of forceps) in order to achieve the required stabilization. Second, during the cutting of the cornea with a knife, pressures induced by the cutting can cause decentration of the eye to occur. The resultant irregular or poorly defined cutting edges can then adversely affect the subsequent healing process and the resultant quality of vision. On the other hand, although the use of laser systems may obviate the adverse effects otherwise caused by unwanted pressures on the eye, the problems of positioning and stabilizing the eye still persist. Thus, in either case, the geometry of the corneal cavity that is prepared to receive the donor graft may be imprecise.

In addition to the difficulties noted above that are encountered while creating a cavity in the cornea of a patient, there is also the problem of creating a donor graft that will have the precise dimensions required to match those of the cavity. In an effort to address this issue for laser procedures, it has been proposed that complimentary masks be made for use with an excimer laser. Specifically, in this case, one mask can be used to create the cavity in the recipient cornea, while its compliment is used to create the graft in the donor cornea. A problem here, however, is the two different mechanical contrivances are used in two separate operations. Further, the stabilization and positioning issues that are inevitably encountered, are not adequately addressed.

In light of the above, it is an objective of the present invention to provide a system and method for performing a corneal transplantation wherein a cavity in the cornea of the patient and the graft from a donor cornea are created using a same laser surgical unit and a same cutting geometry. Another object of the present invention is to provide a system and method for performing a corneal transplantation wherein the cornea of the patient and the donor cornea are each aligned with the surgical laser unit, in the same way during a laser cutting procedure. Still another object of the present invention is to provide a system and method for performing a corneal transplantation that is easy to use, relatively simple to manufacture, and comparatively cost effective.

### SUMMARY OF THE INVENTION

A system for performing a corneal transplantation includes a stationary surgical laser unit having a laser source for directing a laser beam along a beam path. Preferably, the laser beam is an ultra-short pulse laser beam. Additionally, the system of the present invention includes a motorized chair for separately positioning the cornea of a patient and a donor cornea, relative to the laser source. Further, a computer controller is in electronic communication with the motorized chair for moving and reconfiguring the chair.

As contemplated by the present invention, the system includes a mount for holding the donor cornea secure during the corneal transplantation procedure. In one embodiment of the present invention, the mount is configured to hold an entire donor eye, which includes the donor cornea. Alternatively, the mount holds only the donor cornea and the scleral rim of the donor eye. In this alternate embodiment, an artificial anterior chamber is attached to the mount and used to hold the donor cornea and scleral rim secure in the mount. Structurally, the mount is attached to a platform adapter which, in turn, may be mounted on the motorized chair.

In addition to the mount disclosed above, the system includes a stabilizing element of a type as disclosed in co-pending U.S. patent application Ser. No. 10/790,625, which is assigned to the same assignee as the present invention. Importantly, depending on the application, the stabilizing element includes a lens having either an applanating surface or a surface that substantially conforms with the anterior surface of the cornea of the patient. Additionally, the stabilizing element is formed with a vacuum fitting for fixating the stabilizing element to either the cornea of the patient or to the cornea of the donor eye. Along with the stabilizing element, the system of the present invention may include an alignment device which is mounted on the surgical laser unit and is engageable with the stabilizing element. With this interconnection the stabilizing element is aligned with the laser source.

In an alternate embodiment of the present invention, instead of the alignment device and stabilizing element, the system can include an optical assembly for measuring an x-y and a z-position of the donor cornea. Again, the purpose is to align the donor cornea with the surgical laser unit. Structurally, the optical assembly includes an eye tracker for measuring the x-y position of the donor cornea, in accordance with a predetermined orthogonal coordinate system. Additionally, the optical assembly also includes any device, well known in the pertinent art, for measuring the z-position of the donor cornea. For example, the device for measuring the z-position of the cornea may be either a Hartmann-Shack sensor or a confocal microscope.

Preferably, in the operation of the present invention, a donor graft is prepared first and then a cavity for receipt of a donor graft is cut into the cornea of the patient. The dimensions and shape of the cavity are essentially the same as for the donor graft and are well defined. To facilitate the laser cutting of the cornea of the patient, the patient is seated in the chair. Further, the alignment device is mounted or positioned on the surgical laser unit. After the patient is seated in the chair, the motorized chair is moved to generally align the eye of the patient with the surgical laser unit. Once the eye has been generally aligned with the surgical laser unit, the stabilizing element is placed on the anterior surface of the patient's cornea. With the stabilizing element in place, the vacuum device is connected to the stabilizing element, after which the vacuum device is activated. In particular, a vacuum pump is used to create a suction force between the surface of the lens of the stabilizing element and the anterior surface of the cornea. As contemplated by the present invention, the suction force holds the stabilizing element immovable against the eye of the patient.

With the stabilizing element held on the eye of the patient, the chair is reconfigured to move the stabilizing element into an engagement with the alignment device. Once the stabilizing element and alignment device are properly engaged, the eye of the patient is aligned with the laser source. Preferably, the second vacuum device is then activated to create a suction force that maintains the engagement of the stabilizing element with the alignment device. Following the engagement of the stabilizing element and the alignment device, the laser beam is used to remove diseased tissue from the patient's cornea, thereby creating a corneal cavity according to a pre-determined cutting pattern. Specifically, in this operation, the focal point of the laser beam is moved along a predetermined path in the cornea to create a cavity having a specific dimensional configuration. Once the cavity has been created, the engagement between the stabilizing element and the alignment device is terminated, and the patient is moved away from the laser source. The stabilizing element is then removed from the eye of the patient.

Prior to, and in preparation for creating the cavity as disclosed above, the mount is attached to the platform adapter, and the adapter is mounted on the motorized chair. Further, a donor cornea is secured in the mount. As contemplated by the present invention, a stabilizing element is placed on the anterior surface of the donor cornea. Subsequently, the vacuum device is used to fixate the stabilizing element to the donor cornea. It can be appreciated that by using a stabilizing element of the same shape for both the donor cornea and the cornea of the patient, the conformed shapes of the two corneas during photoalteration can be made nearly or substantially the same. In this way, it is possible to ensure that the size and shape of the donor graft precisely matches the size and shape of the corneal cavity.

According to commands sent by the computer controller, the motorized chair is moved to once again engage the stabilizing element with the alignment device. When the eye stabilizing element and alignment device are properly engaged, the donor cornea is aligned with the laser source and a donor graft is cut. Importantly, the cutting pattern for the donor graft generates a graft having a dimensional configuration with dimensions and a shape that will match that of the corneal cavity. Once the donor graft has been cut, the motorized chair is moved away from the laser source, and the stabilizing element is removed from the donor cornea. After the stabilizing element is removed, the donor graft is subsequently placed in an apparatus for transferring the donor graft to the cornea of the patient.

In an alternate embodiment of the present invention, the optical assembly is used to measure the x-y and z-position of the donor cornea prior to creating the donor graft. In this embodiment, neither the stabilizing element nor the alignment device are required. Specifically, the mount is attached to the chair, and the donor cornea is secured in the mount as described above. The motorized chair is then moved to generally align the donor cornea with the laser source. During the alignment procedure, a system operator views the donor cornea through a microscope mounted on the surgical laser unit. Once the system operator determines that the eye is generally aligned with the laser source, the eye tracker is used to measure the x-y position of the donor cornea, according to the predefined orthogonal coordinate system. Additionally, the Hartmann-Shack sensor, or a confocal microscope, measures the z-position of the donor cornea. Once all of the measurements have been taken, the x-y and z-position data is transmitted to the computer controller for processing. Once processed, the data is used by the computer controller to precisely align the laser source with the donor cornea prior to the cutting of the donor graft. Once again, a donor graft having dimensions and a shape precisely matching that of the corneal cavity is cut using a predefined cutting pattern.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a system, in accordance with the present invention, for performing a corneal transplantation;

FIG. 2 is a schematic view of a donor eye positioned in a mount, for presentation of a donor cornea for photoalteration;

FIG. 3 is schematic view of an alternate embodiment of the present invention, for measuring the x-y and z-position of a donor cornea prior to photoalteration of the donor cornea;

FIG. 4A is a perspective view of a cavity in a recipient cornea; and

FIG. 4B is a perspective view of a donor graft cut from a donor cornea for placement in the corneal cavity cut in the cornea of the patient and shown in FIG. 4A.

### DESCRIPTION OF THE PREFERRED EMBODIMENTS

A system for performing corneal transplantations, in accordance with the present invention, is shown in FIG. 1 and is generally designated 10. As shown, the system 10 includes a stationary surgical laser unit 12, which further comprises a laser source 14 for directing a laser beam 16 along a beam path 18. Preferably, the laser beam 16 is an ultra-short pulse laser beam 16 having a wavelength in the range of about 400 nm to 10 μm. Further, the laser beam 16 has a pulse duration in the range of 1 femtosecond to 100 picoseconds, a pulse repetition rate of about 1 to 1000 kHz, and a pulse energy between about 0.1 microjoule and 1 millijoule. Also, it is to be appreciated that an oscillator laser without an additional amplifier can be used. If so, pulse repetition rates of up to 100 MHz can be achieved with pulse energies in a range of 0.1 nanojoule to 10 microjoules.

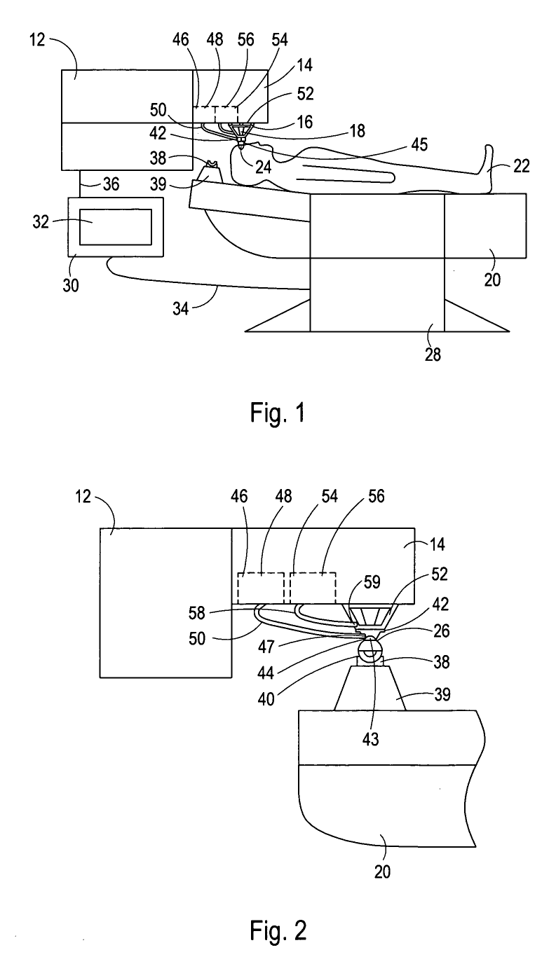

In addition to the laser source 14, the system 10 includes a platform 20 for supporting a patient 22, and for positioning an eye 24 of the patient 22 relative to the laser source 14. As contemplated by the present invention, the platform 20 may also be used to position a donor cornea 26 (FIG. 2) relative to the laser source 14. In the preferred embodiment of the present invention, the platform 20 is a chair that includes a motorized control assembly 28 which can be selectively activated to move and reconfigure the chair 20. A computer controller 30, which has a graphical user interface 32, is in electronic communication with the motorized control assembly 28 for directing the movement of the chair 20. Specifically, an electrical cable 34 interconnects the computer controller 30 and the motorized control assembly 28. Additionally, the computer controller 30 is in electronic communication with the surgical laser unit 12 for controlling the settings, timing and functioning of the unit 12. As shown, an electrical cable 36 connects the computer controller 30 to the surgical laser unit 12.

As can be seen in FIG. 1, the system 10 includes a mount 38 for holding the donor cornea 26. The mount 38, in turn, is affixed to a platform adapter 39, which can be mounted on the chair 20. As can be seen in FIG. 2, the mount 38 can be configured to hold an entire donor eye 40 which includes the donor cornea 26. Additionally, the mount 38 may include an artificial anterior chamber (not shown). Operationally, the artificial anterior chamber is used to secure only the donor cornea 26 and the scleral rim (not shown) of the donor eye 40 in the mount 38.

Cross-referencing FIGS. 1 and 2, it can be seen that the system 10 of the present invention includes a stabilizing element 42. As can be seen in FIG. 2, the stabilizing element 42 includes a lens 44. Importantly, the surface 43 of the lens 44 conforms substantially with the anterior surface of the donor cornea 26 and the cornea 45 of the patient 22. As contemplated by the present invention, the system 10 further includes a vacuum device 46 in fluid communication with a vacuum fitting 47 formed in the stabilizing element 42. More specifically, a vacuum pump 48 is connected to the vacuum fitting 47 via a vacuum line 50.

Still cross referencing FIGS. 1 and 2, the system 10 of the present invention includes an alignment device 52 that is mounted or positioned on the surgical laser unit 12 for engagement with the stabilizing element 42. Specifically, the alignment device 52 may be mounted on the surgical laser unit 12, or the alignment device 52 may be integral to the surgical laser unit 12. Further, as shown, the system 10 includes a vacuum device 54 for maintaining an engagement between the stabilizing element 42 and the alignment device 52, once the two are engaged. Specifically, the vacuum device 54 includes a vacuum pump 56 in fluid communication with a vacuum line 58, which in turn is connected to a vacuum fitting 59 formed in the alignment device 52.

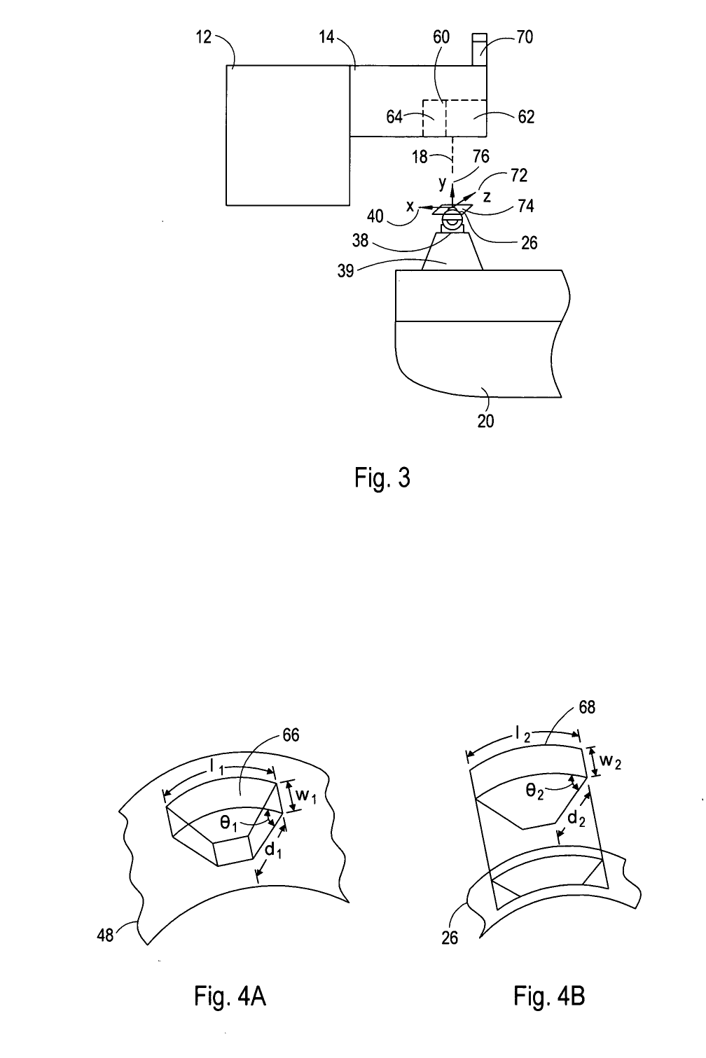

In an alternate embodiment of the present invention, as shown in FIG. 3, the system 10 of the present invention includes an optical assembly 60 for measuring the x-y and z-position of the donor cornea 26. Specifically, the optical assembly 60 includes an eye tracker 62, of a type well known in the pertinent art, for measuring the x-y position of the donor cornea 26. Additionally, the z-position of the donor cornea 26 is measured using a Hartmann-Shack sensor 64 or a confocal detector (not shown).

In the operation of the present invention, a donor graft 68 is prepared and the patient 22 is then positioned in the chair 20 and the stabilizing element 42 is placed on the eye 24 of the patient 22. More specifically, the surface 43 of the lens 44 of the stabilizing element 42 interfaces with the anterior surface of the cornea 45 of the eye 24 of the patient 22. Following commands from the system operator (not shown), the computer controller 30 then directs the motorized control assembly 28 to move and reconfigure the chair 20. Specifically, the chair 20 is moved to generally align the eye 24 of the patient 22 with the stationary surgical laser unit 12. If not already connected, the vacuum line 50 is then connected to both the vacuum fitting 47 of the stabilizing element 42 and to the vacuum pump 48. When activated, the vacuum pump 48 evacuates air from the stabilizing element 42. Consequently, a suction force is created at the interface of the surface 43 of the lens 44 and the anterior surface of the cornea 45 of the eye 24. As envisioned by the present invention, the suction force holds the stabilizing element 42 immovable against the eye 24.

Along with the stabilizing element 42 being placed and held on the eye 24 of the patient 22, the alignment device 52 is mounted, as necessary, on the surgical laser unit 12. Once the alignment device 52 is mounted on the surgical laser unit 12, the chair 20 is moved through a “docking” procedure whereby the stabilizing element 42 is moved to engage with the alignment device 52. When the stabilizing element 42 is properly engaged with the alignment device 52, the eye 24 of the patient 22 is aligned with the surgical laser unit 12. In addition, the eye 24 is positioned at a known distance from the surgical laser unit 12. Thus, when the stabilizing element 42 is engaged with the alignment device 52, the lens 44 and cornea 45 of the eye 24 are a known distance from the cutting lenses (not shown) of the surgical laser unit 12. To ensure that the stabilizing element 42 remains fixedly engaged with the alignment device 52, the vacuum pump 56 is activated to create a suction force whereby the stabilizing element 42 is drawn against the alignment device 52. Once the cornea 45 of the eye 24 of the patient 22 is properly aligned with the laser source 14, the cornea 45 of the eye 24 can be photoaltered to remove diseased tissue from the cornea 45. As can be appreciated by the skilled artisan, removal of diseased tissue creates a cavity for receipt of a donor graft. Referring now to FIG. 4A, it can be seen that a cavity 66 of precise dimensions, of which l₁, d₁, h₁ and θ₁ are only exemplary, is cut by the laser beam 16. The donor graft 68 can now be positioned in the cavity 66 in the cornea 45 of the patient 22.

To create the donor graft 68, for subsequent insertion into the cavity 66, a donor eye 40 is positioned in the mount 38 and the mount 38 is attached to the platform adapter 39, as shown in FIG. 2. The platform adapter 39 is then mounted on the chair 20. Once the mount 38 is attached to the adapter 39, the stabilizing element 42 is placed on the anterior surface of the donor cornea 26. By using a stabilizing element 42 having a same shape with both the donor cornea 26 and the cornea 45 of the patient 22, the anterior surfaces of both corneas 26 and 45 are similarly shaped by the respective lens 44 during photoalteration of the corneas 26 and 45. As such, it is possible to ensure that the size and shape of the donor graft 68 can precisely match the size and shape of the corneal cavity 66. On the other hand, it may be desirable for the donor graft 68 to be customized by the laser (e.g. a slightly larger donor graft 68). In any event, once the stabilizing element 42 is positioned, the vacuum device 46 is employed once again to fixate the stabilizing element 42 to the donor cornea 26.

According to commands sent by the computer controller 30, the motorized chair 20 is moved to once again engage the stabilizing element 42 with the alignment device 52. When the stabilizing element 42 and alignment device 52 are properly engaged, as shown in FIG. 2, the donor cornea 26 is aligned with the laser source 14. Consistent with the procedure that will be subsequently used to create the cavity 66 in the cornea 45 of the patient 22, the vacuum device 54 is employed to maintain the engagement between the stabilizing element 42 and the alignment device 52. Once the donor cornea 26 is properly aligned with the laser source 14, a donor graft 68 is cut from the donor cornea 26 (see FIG. 4B). After the donor graft 68 is cut, the graft 68 is placed in an apparatus (not shown) for transferring the donor graft 68 into the corneal cavity 66. It is an important aspect of the present invention that the dimensions of the donor graft 68 can be substantially the same as the dimensions of the cavity 66 created in the cornea 45 of the patient 22. As indicated above, however, there is flexibility here for the surgeon to customize the size of the donor graft 68. Referring once again to FIG. 4A, it can be appreciated, for example, that l₁=l₂, w₁=w₂, d₁=d₂ and θ₁=θ₂. It should be understood that all of the critical dimensions of the cavity 66 (FIG. 4A) can be substantially the same or slightly smaller than the critical dimensions of the donor graft 68 (FIG. 4B). In this way, the donor graft 68 will fit snugly and precisely within the volume of the cavity 66, thereby aiding the healing process and improving the refractive outcome of the surgery.

Once the cutting of the donor graft 68 is complete, the motorized chair 20 is moved away from the laser source 14, and the stabilizing element 42 is removed from the donor cornea 26. In a subsequent surgical procedure, the donor graft 68 is positioned in the cavity 66 created in the cornea 45 of the patient 22.

In an alternate embodiment of the present invention, the donor cornea 26 is secured in the mount 38 as disclosed above. The chair 20 is then moved and reconfigured to generally align the donor cornea 26 with the laser source 14. As the chair 20 is moving to align the donor cornea 26, the system operator observes the donor cornea 26 through a microscope 70 (FIG. 3) mounted on the surgical laser unit 12. During this procedure, the image of the donor cornea 26 is presented to the system operator on the graphical user interface 32. Using the images presented, the system operator generally aligns the donor cornea 26 with the laser source 14. Once the donor cornea 26 is generally aligned, the optical assembly 60 measures the x-y and z-position of the donor cornea 26, relative to a predefined orthogonal coordinate system 72 (FIG. 3). More specifically, the x-y position of the donor cornea 26 is measured along an x-y plane 74 which is substantially perpendicular to the beam path 18. Additionally, the z-position of the donor cornea 26 is measured along a z-axis 76 which is coincident with the beam path 18. The eye tracker 62 measures the x-y position of the donor cornea 26, and a device such as a Hartmann-Shack sensor 64 or a confocal detector (not shown) measures the z-position of the cornea 26. At the completion of all measurements, the measurement data is communicated electronically to the computer controller 30 via the electrical cable 36, wherein the data is used to align the laser beam 16 with the donor cornea 26. Following this alignment, the donor graft 68 is cut. As described above, the donor graft 68 is then positioned in the cavity 66 previously created in the cornea 45 of the patient 22.

While the particular System for Performing a Corneal Transplantation as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An apparatus for performing a corneal transplantation of a donor graft from a donor cornea into a cavity in a cornea of a patient, wherein the cavity has a predetermined dimensional configuration and the apparatus comprises:
a laser source for generating a laser beam;
a means for presenting a donor cornea with an exposed anterior surface;
a stabilizing element engageable with said laser source to establish a fixed configuration therewith, said stabilizing element having a lens with a surface; and
a means for selectively fixating the surface of said stabilizing element against the anterior surface of the donor cornea, to hold the donor cornea in alignment with said laser source while said laser source is activated to photoalter tissue of the donor cornea and to create the donor graft therefrom, wherein the donor graft has a substantially same dimensional configuration as the cavity for transplantation thereof into the cavity in the cornea of the patient.

2. An apparatus as recited in claim 1 further comprising:
a means for positioning the patient to selectively fixate the surface of said stabilizing element against the anterior surface of the cornea of the patient, to hold the cornea of the patient in alignment with the laser source while said laser source is activated to photoalter tissue for removal of diseased tissue from the cornea of the patient during creation of the cavity for receipt of the donor graft therein; and
a means for transferring the donor graft from the donor cornea into the cavity in the cornea of the patient.

3. An apparatus as recited in claim 1 wherein the surface of said lens substantially conforms with an anterior surface of a cornea of a patient.

4. An apparatus as recited in claim 3 wherein said presenting means is a mount and said mount includes an artificial anterior chamber for fixedly holding only the donor cornea and a scleral rim of a donor eye, after the donor cornea and the scleral rim are surgically removed from the donor eye.

5. An apparatus as recited in claim 1 wherein said laser source is a femtosecond laser source.

6. An apparatus as recited in claim 1 wherein said stabilizing element is formed with a vacuum fitting, and further wherein the fixating means of the apparatus comprises:
a vacuum line connected to said vacuum fitting; and
a vacuum pump in fluid communication with said vacuum line for generating a suction force to fixate the surface of said stabilizing element against the anterior surface of the donor cornea.

7. An apparatus as recited in claim 2 wherein said positioning means is a chair having a motorized control assembly for reconfiguring and moving said chair.

8. An apparatus as recited in claim 1 further comprising an alignment device mounted on said surgical laser unit for engaging with said stabilizing element, to establish the fixed configuration between said stabilizing element and said laser source.

9. A method for performing a corneal transplantation of a donor graft from the donor cornea into a cavity in a cornea of a patient, wherein the cavity has a predetermined dimensional configuration and the method comprises the steps of:
presenting a donor cornea having an exposed anterior surface;
fixating a stabilizing element against the anterior surface of the donor cornea, said stabilizing element having a lens with a surface;
engaging a laser source with said stabilizing element to align the donor cornea with said laser source; and
activating said laser source to photoalter tissue of the donor cornea and to create the donor graft therefrom, wherein the donor graft has a substantially same dimensional configuration as the cavity for transplantation thereof into the cavity in the cornea of a patient.

10. A method as recited in claim 9 further comprising the steps of:
positioning the patient to selectively fixate the surface of a stabilizing element against the anterior surface of the cornea of the patient, to hold the cornea of the patient in alignment with said laser source while said laser source is activated to photoalter tissue for removal of diseased tissue from the cornea of the patient during creation of the cavity for receipt of the donor graft therein; and
transferring the donor graft from the donor cornea into the cavity in the cornea of the patient.

11. A method as recited in claim 9 wherein said presenting step further comprises the steps of:
fixedly positioning the donor cornea in a mount;
attaching said mount to a platform adapter;
mounting said platform adapter on a motorized chair; and
reconfiguring and moving said chair to present the donor cornea.

12. A method as recited in claim 11 wherein said positioning step further comprises the steps of:
securing the donor cornea and a scleral rim of a donor eye in an artificial anterior chamber, wherein the donor cornea and the scleral rim are surgically removed from the donor eye prior to securing the donor cornea and scleral rim in the artificial anterior chamber; and
attaching said artificial anterior chamber to said mount for fixedly positioning the donor cornea in said mount.

13. A method as recited in claim 9 wherein said stabilizing element is formed with a vacuum fitting, and wherein said fixating step further comprises the steps of:
attaching a vacuum line to said vacuum fitting; and
activating a vacuum pump in fluid communication with said vacuum line for generating a suction force to fixate the surface of said stabilizing element against the anterior surface of the donor cornea.

14. A method as recited in claim 9 wherein said laser source is a femtosecond laser source.

15. A system for performing a corneal transplantation which comprises:
a laser source for generating a laser beam;
a mount for holding a donor cornea in alignment with said laser source;
a means for positioning the cornea of a patient in alignment with the laser source while said laser beam is focused along a predetermined path to photoalter tissue in the cornea of the patient to create a cavity having a predetermined dimensional configuration;
a means for focusing the laser beam to a successive plurality of focal points along a substantially same predetermined path to photoalter tissue of the donor cornea and to create a donor graft therefrom having a substantially same predetermined dimensional configuration as the cavity; and
a means for transferring the donor graft from the donor cornea into the cavity in the cornea of the patient.

16. A system as recited in claim 15 which further comprises:
a stabilizing element engageable with said laser source to establish a fixed configuration therewith, said stabilizing element having a lens with a surface; and
a means for selectively fixating the surface of said stabilizing element against the anterior surface of the donor cornea, to hold the donor cornea in alignment with said laser source while said laser source is activated to photoalter tissue of the donor cornea.

17. A system as recited in claim 15 wherein said determining means comprises:
a means for measuring an x-y position of the donor cornea, according to a predefined orthogonal coordinate system, wherein the x-y plane of the coordinate system is substantially perpendicular to the beam path; and
a means for measuring a z-position of the donor cornea, according to the predefined orthogonal coordinate system, wherein the z-axis of the coordinate system is substantially coincident with the beam path.

18. A system as recited in claim 17 wherein said means for measuring a z-position is a device selected from the group consisting of a Hartmann-Shack sensor and a confocal microscope.

19. A system as recited in claim 15 wherein said positioning means is a chair having a motorized control assembly for reconfiguring and moving said chair.

20. A system as recited in claim 15 wherein said laser source generates a femtosecond laser beam.
